SEQUENCE LISTING (5 sequences)

SEQ ID NO: 1 - protein, 1070 aa, HOMO SAPIENS
MCFSFIMPPAMADILDIWAVDSQIASDGSIPVDFLLPTGIYIQLEVPREATISYIKQMLWKQVHNYPMFNLLMDIDSYMFACVNQTAVYEELEDETRRLCDVRPFLPVLKLVTRSCDPGEKLDSKIGVLIGKGLHEFDSLKDPEVNEFRRKMRKFSEEKILSLVGLSWMDWLKQTYPPEHEPSIPENLEDKLYGGKLIVAVHFENCQDVFSFQVSPNMNPIKVNELAIQKRLTIHGKEDEVSPYDYVLQVSGRVEYVFGDHPLIQFQYIRNCVMNRALPHFILVECCKIKKMYEQEMIAIEAAINRNSSNLPLPLPPKKTRIISHVWENNNPFQIVLVKGNKLNTEETVKVHVRAGLFHGTELLCKTIVSSEVSGKNDHIWNEPLEFDINICDLPRMARLCFAVYAVLDKVKTKKSTKTINPSKYQTIRKAGKVHYPVAWVNTMVFDFKGQLRTGDIILHSWSSFPDELEEMLNPMGTVQTNPYTENATALHVKFPENKKQPYYYPPFDKIIEKAAEIASSDSANVSSRGGKKFLPVLKEILDRDPLSQLCENEMDLIWTLRQDCREIFPQSLPKLLLSIKWNKLEDVAQLQALLQIWPKLPPREALELLDFNYPDQYVREYAVGCLRQMSDEELSQYLLQLVQVLKYEPFLDCALSRFLLERALGNRRIGQFLFWHLRSEVHIPAVSVQFGVILEAYCRGSVGHMKVLSKQVEALNKLKTLNSLIKLNAVKLNRAKGKEAMHTCLKQSAYREALSDLQSPLNPCVILSELYVEKCKYMDSKMKPLWLVYNNKVFGEDSVGVIFKNGDDLRQDMLTLQMLRLMDLLWKEAGLDLRMLPYGCLATGDRSGLIEVVSTSETIADIQLNSSNVAAAAAFNKDALLNWLKEYNSGDDLDRAIEEFTLSCAGYCVASYVLGIGDRHSDNIMVKKTGQLFHIDFGHILGNFKSKFGIKRERVPFILTYDFIHVIQQGKTGNTEKFGRFRQCCEDAYLILRRHGNLFITLFALMLTAGLPELTSVKDIQYLKDSLALGKSEEEALKQFKQKFDEALRESWTTKVNWMAHTVRKDYRS

SEQ ID NO: 2 - protein, 48 aa, HOMO SAPIENS
FDKIIEKAAEIASSDSANVSSRGGKKFLPVLKEILDRDPLSQLCENEM

SEQ ID NO: 3 - protein, 621 aa, HOMO SAPIENS
VPHGLEDLLNPIGVTGSNPNKETPCLELEFDWFSSVVKFPDMSVIEEHANWSVSREAGFSYSHAGLSNRLARDNELRENDKEQLKAISTRDPLSEITEQEKDFLWSHRHYCVTIPEILPKLLLSVKWNSRDEVAQMYCLVKDWPPIKPEQAMELLDCNYPDPMVRGFAVRCLEKYLTDDKLSQYLIQLVQVLKYEQYLDNLLVRFLLKKALTNQRIGHFFFWHLKSEMHNKTVSQRFGLLLESYCRACGMYLKHLNRQVEAMEKLINLTDILKQEKKDETQKVQMKFLVEQMRRPDFMDALQGFLSPLNPAMQLGNLRLEECRIMSSAKRPLWLNWENPDIMSELLFQNNEIIFKNGDDLRQDMLTIQIIRIMENIWQNQGLDLRMLPYGCLSIGDCVGLIEVVRNSHTIMQIQCKGGLKGALQFNSHTLHQWLKDKNKGEIYDAAIDLFTRSCAGYCVATFILGIGDRHNSNIMVKDDGQLFHIDFGHFLDHKKKKFGYKRERVPFVLTQDFLIVISKGAQECTKTREFERFQEMCYKAYLAIRQHANLFINLFSMMLGSGMPELQSFDDIAYIRKTLALDKTEQEALEYFMKQMNDAHHGGWTTKMDWIFHTIKQHALN

SEQ ID NO: 4 - protein, 607 aa, HOMO SAPIENS
  misc_feature (607)..(607): Xaa can be any naturally occurring amino acid
FPDELEEMLNPMGTVQTNPYTENATALHVKFPENKKQPYYYPPFDKIIEKAAEIASSDSANVSSRGGKKFLPVLKEILDRDPLSQLCENEMDLIWTLRQDCREIFPQSLPKLLLSIKWNKLEDVAQLQALLQIWPKLPPREALELLDFNYPDQYVREYAVGCLRQMSDEELSQYLLQLVQVLKYEPFLDCALSRFLLERALGNRRIGQFLFWHLRSEVHIPAVSVQFGVILEAYCRGSVGHMKVLSKQVEALNKLKTLNSLIKLNAVKLNRAKGKEAMHTCLKQSAYREALSDLQSPLNPCVILSELYVEKCKYMDSKMKPLWLVYNNKVFGEDSVGVIFKNGDDLRQDMLTLQMLRLMDLLWKEAGLDLRMLPYGCLATGDRSGLIEVVSTSETIADIQLNSSNVAAAAAFNKDALLNWLKEYNSGDDLDRATEEFTLSCAGYCVASYVLGIGDRHSDNIMVKKTGQLFHIDFGHILGNFKSKFGIKRERVPFILTYDFIHVIQQGKTGNTEKFGRFRQCCEDAYLILRRHGNLFITLFALMLTAGLPELTSVKDIQYLKDSLALGKSEEEALKQFKQKFDEALRESWTTKVNWMAHTVRKDYRSX

SEQ ID NO: 5 - protein, 601 aa, HOMO SAPIENS
VPDEKGELLNPTGTVRSNPNTDSAAALLICLPEVAPHPVYYPALEKILELGRHSECVHVTEEEQLQLREILERRGSGELYEHEKDLVWKLRHEVQEHFPEALARLLLVTKWNKHEDVAQMLYLLCSWPELPVLSALELLDFSFPDCHVGSFAIKSLRKLTDDELFQYLLQLVQVLKYESYLDCELTKFLLDRALANRKIGHFLFWHLRSEMHVPSVALRFGLILEAYCRGSTHHMKVLMKQGEALSKLKALNDFVKLSSQKTPKPQTKELMHLCMRQEAYLEALSHLQSPLDPSTLLAEVCVEQCTFMDSKMKPLWIMYSNEEAGSGGSVGIIFKNGDDLRQDMLTLQMIQLMDVLWKQEGLDLRMTPYGCLPTGDRTGLIEVVLRSDTIANIQLNKSNMAATAAFNKDALLNWLKSKNPGEALDRAIEEFTLSCAGYCVATYVLGIGDRHSDNIMIRESGQLFHIDFGHFLGNFKTKFGINRERVPFILTYYDFVHVIQQGKTNNSEKFERFRGYCERAYTILRRHGLLFLHLFALMRAAGLPELSCSKDIQYLKDSLALGKTEEEALKHFRVKFNEALRESWKTKVNWLAHNVSKDNRQ

(19) United States
(12) Patent Application Publication
(10) Pub. No.: US 2014/0113869 A1
(43) Pub. Date: Apr. 24, 2014

(54) Gbetagamma BINDING SITE ON THE PIK3CB GENE PRODUCT AND METHODS OF USE

(71) Applicants: Jonathan M. Backer, New Rochelle, NY (US); Hashem A. Dbouk, Bronx, NY (US)

(72) Inventors: Jonathan M. Backer, New Rochelle, NY (US); Hashem A. Dbouk, Bronx, NY (US)

(73) Assignee: Albert Einstein College of Medicine of Yeshiva University, Bronx, NY (US)

(21) Appl. No.: 14/124,839

(22) PCT Filed: Jun. 12, 2012
(86) PCT No.: PCT/US2012/042040

§ 371 Date: Dec. 9, 2013

Related U.S. Application Data
(60) Provisional application No. 61/496,282, filed on Jun. 13, 2011.

(51) Int. Cl. C12N 9/12

(52) U.S. Cl. CPC C12N 9/1205
USPC 514/19.5; 514/19.3; 514/44 R; 435/15

(57) ABSTRACT

Methods of treating a disease in a subject are provided comprising administering to the subject an amount of an agent which reduces, or prevents, interaction of a Gβγ with a pi 110β effective to treat the disease. Methods are also provided for identifying an inhibitor of interaction between a Gβγ and a ρ110β. Compositions are provided comprising a peptide comprising amino acid residues having the KAAEIASSDSANVSSRGGKKFLPV (SEQ ID NO:6).

### CROSS-REFERENCE TO RELATED APPLICATIONS

This application claims benefit of U.S. Provisional Application No. 61/496,282, filed Jun. 13, 2011, the contents of which are hereby incorporated by reference.

### STATEMENT OF GOVERNMENT SUPPORT

This invention was made with government support under grant numbers GM55692 and PO1 CA 100324 awarded by the National Institutes of Health. The government has certain rights in the invention.

### BACKGROUND OF THE INVENTION

Throughout this application various publications are referred to in parentheses. The disclosures of these publications, and of all patents, patent application publications and books referred to herein, are hereby incorporated by reference in their entirety into the subject application to more fully describe the art to which the subject invention pertains.

Signaling by Class I Phosphoinositide 3-kinases (PI3Ks) is commonly up regulated in tumors by gene amplification, by activating mutations, or by inactivation of PTEN, a tumor suppressor lipid phosphatase. Class I Phosphoinositide 3-kinases (PI3Ks) produce phosphatidylinositol (3,4,5)P3 (PIP3) in cells and regulate proliferation, survival, and motility. They are obligate heterodimers consisting of distinct catalytic (p110) subunits bound to the same regulatory (p85) subunits. Among the three Class IA PI 3-kinases, the PIK3CB gene product p110β is unique, because it can be activated both by Receptor Tyrosine Kinases (RTKs) and downstream of G-protein-coupled receptors (GPCRs) via direct binding to Gβγ heterodimers. PTEN-deficient prostate cancer development specifically depends on PI3Kβ activity, but the mechanism for this specificity is currently unknown. Whether GPCRs have a role in PI3Kβ-mediated transformation of PTEN-null cells has remained an open question, because of the lack of tools to specifically probe the Gβγ-PI3Kβ interaction. Defining the role of Gβγ in activating effectors such as p110β is challenging, due to the transient nature of their interactions and due to a lack of a distinct Gβγ-binding motif that would identify its target binding sites. This contrasts with the mechanism of activation of PI3Ks by RTKs, which involve h e affinity interactions that have been well characterized. To investigate the mechanism of p110β activation downstream of GPCRs by Gβγ, and to define the role of this interaction in p110β signaling in vivo, the Gβγ binding site on p lop has been investigated.

The present invention identifies the regulation of p110β and p110γ by GPCRs and provides therapies and assays based thereon.

### SUMMARY OF THE INVENTION

A method of treating a disease in a subject is provided comprising administering to the subject an amount of an agent which reduces, or prevents, interaction of a Gβγ with a p110β effective to treat the disease.

Also provided is as method for identifying a candidate agent as an inhibitor of Gβγ activation of p110β comprising contacting a p110β with the candidate agent in the presence of Gβγ under conditions permitting the Gβγ to activate the p110β, wherein reduced activation of p110β by Gβγ in the presence of the candidate agent compared to activation of p110β by Gβγ in the absence of the candidate agent under conditions permitting the Gβγ to activate the pi top indicates that the candidate agent is an inhibitor of Gβγ activation of p110β.

Also provided is a method for inhibiting Gβγ activation of p110β without inhibiting lipid kinase activity of p110β comprising contacting the p110β with an agent that reduces or prevents interaction of Gβγ with the p110β without inhibiting lipid kinase activity of p110β.

Also provided is a method of identifying an inhibitor of interaction between a Gβγ and a p110β, the method comprising a) modeling in silico the 3-dimensional site or sites on Gβγ which bind KAAEIASSDSANVSSRGGKKFLPV (SEQ ID NO:6), b) testing in silica if a compound from a library of compounds binds to the modeled 3-dimensional site or sites, and c) determining in vitro if a chemically stable small molecule identified as binding to the site or sites in silico in b) inhibits the interaction interaction between a Gβγ and a p110β, wherein a chemically stable small molecule that inhibits the interaction between a Gβγ and a p110β is identified as art inhibitor.

A method is also provided of inhibiting proliferation and/or chemotaxis of a PTEN-null tumor cell comprising contacting the PTEN-null tumor cell with an amount of an agent which reduces, or prevents, interaction of a Gβγ with a p110β effective to inhibit proliferation and/or chemotaxis of the PTEN-null tumor cell.

### BRIEF DESCRIPTION OF THE DRAWINGS

FIG. 1. Top: Domain structure of Chimera 2. Bottom. Akt activation in cells expressing p85 plus p110α, p110β or Chimera 2, without or with Gβγ.

FIG. 2. C2-helical linker position in p110β (p110delta is SEQ ID NO:1 and p110beta is SEQ ID NO:2).

FIG. 3A-3C. (3A) Basal lipid kinase activity of wild type or mutant p110β. (3B) Activation of wild type or mutant p110β by tyrosine phosphopeptides. (3C) Activation of wild type p110α or wild type or mutant p110β by recombinant Gβγ.

FIG. 4A-4B. (4A): Akt activation in cells transfected with p85 and wild type or mutant p110β (⁵³²KK-DD), without or with Gβγ. (4B): Colony formation in soft agar by cells transfected as above. (4C): Foci formation by cells transfected as above. (D). Migration in a wound closure assay cells transfected as above.

FIG. 5. Percent stimulation of p85/p110β dimers incubated without or with Gβγ in the absence or presence of 5-1Old excess peptide. Pep=p110β peptide. Scram=scrambled peptide control.

FIG. 6A-6B. (6A) NIH3T3 cells were transfected without or with Gβγ and incubated with scrambled (control) or p110β peptide. pT308-Akt was measured by blotting. (6B) Cell transfected as above were incubated with myristoylated peptide, TAT-tagged peptide, or unmodified peptide. pT308-Akt was measured by blotting.

FIG. 7A-7B. (7A). NIH3T3 cells were transfected without or with p85/p110β and incubated without or with 30 μM p110β peptide or scrambled control peptide. Colony formation in soft agar by cells transfected and treated with peptide as above. (7B). Foci were measured after 2 weeks.

FIG. 8. NIH3T3 cells were transfected without or with p85/p110β. Confluent monolayers were scratched with a pipette tip, and wound closure after 24h was measured in the absence or presence of 30 μM p110β peptide or scrambled control peptide

FIG. 9A-9D. Peptide inhibitors of Gβγ -mediated p110β/p85 activation are specific for p85/p110β. (9A) Myristoylated p110β peptides or SIGK peptide were preincubated with biotinylated Gβγ or 5 min, followed by addition of phage particles displaying the SIGK peptide, After a 1 h incubation at room temperature, Anti-M-13 phase monoclonal antibody was added followed by addition of Streptavidin coated Alphascreen donor beads and protein A coated Alphascreen acceptor beads. After a 2 h incubation, the Alphascreen signal was read on a Perkin Elmer Envision Multilabel Plate reader. (9B) Recombinant p110β/p85 (Inners were produced in HEK293T cells and assayed in the absence or presence of Gβγ and p110β-derived peptide (30 μM) or SIGK peptide (10 μM). (9C) HEK293E cells were transfected with p101/p110γ without or with Gβγ. Cells were treated with myristoylated p110β-derived peptide or scrambled peptide, and assayed for pT308-Akt levels by western blot. The data are the mean −/+SD from 2 separate experiments. (9D) Membranes from Sf9 cells expressing recombinant adenylyl cyclase were incubated for 10 min at 30° C. with 20 nM Gsα, without or with 20 nM Gβγ and a known inhibitor peptide (QEHA;(6)) or myristoylated p110β peptide (30 μM). The data are means −/+SD from duplicates, and are representative of two separate experiments. (9E) HEK293T cells were transfected with p85α and p110β, incubated with wild typre or scrambled myristoylated p110β peptide, and cell lysates were incubated with immobilized GST or GST-Rab5. Bound proteins were analyzed by western blot. The data are the mean −/+SD from two separate experiments. (9F) HEK293A cells expressing GFP-LC3 were incubated in complete media or in PBS containing 100 nM rapamycin and wild typre or scrambled myristoylated p110β peptide for 2 h. The cells were fixed and the number of GFP punctae per cell was counted using a Nikon Eclipse 400 microscope with 60× 1.4 N.A. optics. The data are normalized to the number of punctae in DMSO-treated cells, and are the mean −/+SEM from three separate experiments.

FIG. 10A-10C. Inhibition of prostate cancer cell proliferation and chemotaxis: (A) Proliferation of PC-3 cells was measured by the MTS assay in the absence or presence of 30 μM myristoylated p110β-derived peptide or scrambled peptide. (B) Proliferation assays were performed on two PTEN-null endometrial cancer cell lines (AN3CA and RL95-2) and one PTEN positive endometrial cell line (KLE) grown in the absence or presence of myristoylated p110β-derived peptide or scrambled peptide. (C) PC-3 cells chemotaxis toward 10% PBS in the absence or presence of 20 μM p110β-derived peptide or scrambled peptide was measured in Boyden chambers.

FIG. 11A-11B. Comparison of p110 helical/kinase domains (alpha is SEQ ID NO:3; beta is SEQ ID NO:4, delta is SEQ ID NO:5).

### DETAILED DESCRIPTION OF THE INVENTION

A method of treating a disease in a subject is provided comprising administering to the subject an amount of an agent which reduces, or prevents, interaction of a Gβγ with a p110β effective to treat the disease.

In an embodiment, the disease is a cancer. In an embodiment, the cancer is a prostate cancer, a glioma, a breast cancer, an H-Ras driven tumor, a transforming growth factor beta (TGFβ)-dependent tumor, a c-Kit-dependent cancer, an endometrial cancer, or acute promyelocytic leukemia. In an embodiment, the cancer is a c-Kit-dependent cancer and is a testicular cancer. In an embodiment, the cancer is a prostate cancer, a glioma, a breast cancer, an H-Ras driven tumor, a transforming growth factor beta (TGFβ) dependent tumor, a c-Kit-dependent cancer or acute promyelocytic leukemia. In an embodiment, the cancer is a prostate cancer or a glioblastoma or endometrial cancer. In an embodiment, the cancer is phosphatase and tensin homolog (PTEN) null.

In an embodiment the agent is a peptide comprising amino acid residues having the same sequence as, or the same sequence as an active portion of, residues 513 to 537 of SEQ ID NO:1. In an embodiment, the peptide is acylated or is myristoylated. An active portion of residues 513 to 537 of SEQ ID NO:1 is a portion of residues 513 to 537 of SEQ ID NO:1 which is capable of inhibiting interaction of a Gβγ with a p110β. In an embodiment the peptide is 25 amino acids in length. In an embodiment the peptide is 26 amino acids in length. In an embodiment the peptide is 27 amino acids in length. In an embodiment the peptide is 28 amino acids in length. In an embodiment the peptide is 29 amino acids in length. In an embodiment the peptide is 30 amino acids in length. In an embodiment the peptide is 31-35 amino acids in length, In an embodiment the peptide is 36-40 amino acids in length. In an embodiment the peptide is 41-45 amino acids in length. In an embodiment the peptide is 46-50 amino acids in length.

In an embodiment, the agent is an oligonucleotide which reduces or blocks the binding of the Gβγ to the p110β. In an embodiment, the agent is an aptamer, a nucleic acid, an oligonucleotide, a small organic molecule of 2000 Daltons or less, a small organic molecule of 1000 Daltons or less, or a nucleic-acid effector of RNAi in an embodiment, the agent is a nucleic-acid effector of RNAi and is a shRNA, or siRNA. In an embodiment, the anent is attached to a moiety that renders it cell-permeable. Such moieties are well known in the art, for example, penetratin, an antennapedia peptide (RQIKIWFQNRRMKWKK-NH₂) See also Carrigan C N, Imperiali B., The engineering of membrane-permeable peptides, Anal Biochem. 2005 Jun. 15; 341(2):290-8.

In an embodiment, the agent comprises a cDNA encoding a stable inert protein, wherein (a) a peptide having the sequence of residues 513 to 537 of SEQ ID NO:1 or (b) as peptide having the sequence KAAEIASSDSANVSSRGGKKFLPV (SEQ ID NO:6) is attached via a peptide bond to the C-terminus of the stable inert protein, to the N-terminus of the stable inert protein, or (a) a peptide having the sequence of residues 513 to 537 of SEQ ID NO:1 or (b) two peptides, each having the sequence KAAEIASSDSANVSSRGGKKFLPV (SEQ ID NO:6) are attached to the stable inert protein, one to the C-terminus and one to the N-terminus of the stable inert protein. In an embodiment, the agent is introduced into cells of the subject by transduction, lentiviral delivery or adenoviral delivery, in an embodiment, the stable inert protein is a thiredoxin or a small ubiquitin-like modifier (SUMO).

In an embodiment, the agent binds to the C2 domain helical linker of p110β.

In an embodiment, the agent binds to a portion of Gβγ which binds to the C2 domain helical linker of p110β.

In an embodiment, the agent is a peptide comprising amino acid residues having the same sequence as residues 513 to 537 of SEQ NO:1 or has the sequence KAAEIASSDSANVSSRGGKKFLPV (SEQ ID NO:6).

In an embodiment, the agent is an oligonucleotide which reduces or blocks the binding of the Gβγ to the p110β.

In an embodiment, the disease is thrombosis, fragile X syndrome or inflammation.

Also provided is a method for identifying a candidate agent as an inhibitor of Gβγ activation of p110β comprising contacting a p110β with the candidate agent in the presence of Gβγ under conditions permitting the Gβγ to activate the p110β, wherein reduced activation of p110β by Gβγ in the presence of the candidate agent compared to activation of p110β by Gβγ in the absence of the candidate agent under conditions permitting the Gβγ to activate the p110β indicates that the candidate agent is an inhibitor of Gβγ activation of p110β.

In an embodiment, the candidate agent is a peptide, an aptamer, a nucleic acid, an oligonucleotide., or a small organic molecule of 2000 daltons or less or of 100 daltons or less.

Also provided is a method for inhibiting Gβγ activation of p110β without inhibiting, lipid kinase activity of p110β comprising contacting the p110β with an agent that reduces or prevents interaction of Gβγ with the p110β without inhibiting lipid kinase activity of p110β.

In an embodiment, the p110β contacted with the Gβγ is activatable by receptor tyrosine kinases.

In an embodiment, the agent is a peptide comprising amino acid residues having the KAAEIASSDSANVSSRGGKKFLPV (SEQ ID NO:6).

In an embodiment, the agent is oligonucleotide which reduces or blocks the binding of the Gβγ to the p110β.

In an embodiment, the agent binds to the site on p110β to which Gβγ binds.

In an embodiment, the agent binds to residues 513 to 537 of SEQ ID NO:1.

In an embodiment, the agent does not bind to the ATP-binding site on p110β.

In an embodiment, the agent does not bind to the catalytic site of p110β.

In an embodiment, the agent binds to the C2 domain helical linker of p110β.

Also provided is a method of identifying an inhibitor of interaction between a Gβγ and a p110β, the method comprising a) modeling in silico the 3-dimensional site or sites on Gβγ which bind(s) KAAEIASSDSANVSSRGGKKFLPV (SEQ ID NO:6), b) testing in silico if a compound from a library of compounds binds to the modeled 3-dimensional site or sites, and c) determining in vitro if a chemically stable small molecule identified as binding to the site or sites in silico b) inhibits the interaction interaction between a Gβγ and a p110β, wherein a chemically stable small molecule that inhibits the interaction between a Gβγ and a p110β is identified as an inhibitor. In silico modeling of 3-D binding sites for rational drug design is known in the art. For example, see Computational Resources for Protein Modeling and Drug Discovery Applications, Infectious Disorders—Drug Targets (2009), 9, 557-562, B. Dhaliwal and Y. W. Chen, the contents of which are hereby incorporated by reference.

An apparatus system for identifying an inhibitor of interaction between a Gβγ and a p110β comprising:

one or more data processing apparatus and a computer-readable medium coupled to the one or more data processing apparatus having instructions stored thereon which, when executed by the one or more data processing apparatus, cause the one or more data processing apparatus to perform a method comprising a) modeling in silico the 3-dimensional site or sites on Gβγ which bind KAAEIASSDSANVSSRGGKKFLPV (SEQ ID NO:6), and b) testing in silico if a compound from a library of compounds binds to the modeled 3-dimensional site or sites, wherein a small molecule that binds to the modeled 3-dimensional site or sites in silico is identified as an inhibitor of the interaction between a Gβγ and a p110β.

In an embodiment of the inventions described herein, the site on Gβγ which binds KAAEIASSDSANVSSRGGKKFLPV (SEQ ID NO:6) is a β-propeller region.

A method is also provided of inhibiting proliferation and/or chemotaxis of a PTEN-null tumor cell comprising contacting the PTEN-null tumor cell with an amount of an agent which reduces, or prevents, interaction of a Gβγ with a p110β effective to inhibit proliferation and/or chemotaxis of the PTEN-null tumor cell.

“Treating” a cancer as used herein means effecting a state where one or more measurable symptoms of the disease, such as the progression of the cancer itself, size of a tumor of the cancer, or other parameter(s) by which the disease is characterized, is or are reduced, ameliorated, prevented, placed in a state of remission, or maintained in a state of remission.

As used herein, a “cancer” is a disease state well-recognized in the medical field characterized by the presence in a subject of cells demonstrating abnormal uncontrolled replication,

In an embodiment, the oligonucleotide referred to herein as an agent which reduces or prevents the interaction of Gβγ with p110β, is an aptamer which is a single-stranded oligonucleotide or oligonucleotide analog that binds to a particular target molecule, such as a Gβγ or p110β, or to a nucleic acid encoding a Gβγ or p110β. and inhibits the function or expression thereof, as appropriate, in an embodiment, the aptamer is an oligoribonucleotide. Alternatively, an “aptamer” may be a protein aptamer which consists of a variable peptide loop attached at both ends to a protein scaffold that interferes with the interaction of Gβγ with p110β.

The agent can be administered to the subject M a pharmaceutical composition comprising a pharmaceutically acceptable carrier. Examples of acceptable pharmaceutical carriers include, but are not limited to, additive solution-3 (AS-3), saline, phosphate buffered saline, Ringer's solution, lactated Ringer's solution, Locke-Ringer's solution, Krebs Ringer's solution, Hartmann's balanced saline solution, and heparinized sodium citrate acid dextrose solution. The pharmaceutically acceptable carrier used can depend on the route of administration. The pharmaceutical composition can be formulated for administration by any method known in the art, including but not limited to, systemic administration, oral administration, parenteral administration, intravenous administration, transdermal administration, intranasal administration, and administration through an osmotic mini-pump. The compounds can be applied to the skin, for example, in compositions formulated as skin creams, or as sustained release formulations or patches.

In an embodiment, the agent is introduced directly into the site of the cancer, e.g. into a tumor of the cancer by, for example, injection or cannulation.

The agents and compositions of this invention may be administered in various forms, including those detailed herein. The treatment with the agent may be a component of a combination therapy or an adjunct therapy, i.e. the subject or patient in need of the agent is treated or given another drug for the disease in conjunction with one or more of the instant compounds. This combination therapy can be sequential therapy where the patient is treated first with one agent and then the other drug or the two are given simultaneously. These can be administered independently by the same route or by two or more different routes of administration depending on the dosage forms employed.

As used herein, a “pharmaceutically acceptable carrier” is a pharmaceutically acceptable solvent, or suspending vehicle, for delivering the instant agents to an animal or to a human. The carrier may be liquid or solid and is selected with the planned manner of administration in mind. Liposomes are also a pharmaceutically acceptable carrier.

The dosage of the agent administered in treatment will vary depending upon factors such as the pharmacodynamic characteristics of a specific chemotherapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of the agent may comprise a single compound or mixtures thereof with anti-cancer compounds, or tumor growth inhibiting compounds. The agents can be administered in oral dosage forms as tablets, capsules, pills, powders, granules, elixirs, tinctures, suspensions, syrups, and emulsions. The agents may also be administered in intravenous (bolus or infusion), intraperitoneal, subcutaneous, or intramuscular form, or introduced directly, e.g. by injection or other methods, into the cancer, all using dosage forms well known to those of ordinary skill in the pharmaceutical arts.

The agents can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit can be in a form suitable for, in non-limiting examples, oral, rectal, topical, intravenous or direct injection or parenteral administration. The compounds can be administered alone but are generally mixed with a pharmaceutically acceptable carrier. This carrier can be a solid or liquid, and the type of carrier is generally chosen based on the type of administration being used. In one embodiment the carrier can be a monoclonal antibody. The active agent can be coadministered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, (diluents, disintegrating agents, coloring agents, flavoring agents, flow-inducing agents, and melting agents. Examples of suitable liquid dosage forms include solutions or suspensions n water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms nay also include minerals and other materials to make them compatible with the type of injection or delivery system chosen.

Specific examples of pharmaceutical acceptable carriers and excipients that may be used to formulate oral dosage forms of the agents used in the present invention are described in U.S. Pat. No. 3,903,297 to Robert, issued Sep. 2, 1975. Techniques and compositions for making dosage forms useful in the present invention are described-in the following references: 7 Modem Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976) Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences Vol 61 (Alain Rolland, Ed., 1993); Drug Delivery to the Gastrointestinal Tract (Ellis Horwood Books in the Biological Sciences. Series in Pharmaceutical Technology; J. G. Hardy., S. S, Davis, Clive G. Wilson Eds.); Modem Pharmaceutics Drugs and the Pharmaceutical Sciences, Vol 40 (Gilbert S. Banker, Christopher T. Rhodes, Eds.). All of the aforementioned publications are incorporated by reference herein.

Tablets comprising the agents used may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate carboxymethylcellulose, polyethylene glycol, waxes, and the like, Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The agents can also be administered in the form of liposome delivery systems, such as small unilamellar vesicles, large unilamallar vesicles, and multilamellar vesicles. Liposomes can be formed from a variety of phospholipids, such as cholesterol, stearylamine, or phosphatidylcholines. The compounds may be administered as components of tissue-targeted emulsions.

The agents may also he coupled to soluble polymers as targetable drug carriers or as a prodrug. Such polymers include polyvinylpyrrolidone, pyran copolymer, polyhydroxylpropylmethacrylamide-phenol, polyhydroxyethylasparta-midephenol, or polyethyleneoxide-polylysine substituted with palmitoyl residues. Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block, copolymers of hydrogels.

The agents can be administered orally in solid dosage forms, such as capsules, tablets, and powders, or in liquid dosage forms, such as elixirs, syrups, and suspensions. They can also be administered parentally, in sterile liquid dosage forms.

Gelatin capsules may contain the active ingredient compounds and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract,

For oral administration in liquid dosage form, the oral drug components are combined with any oral, non-toxic, pharmaceutically acceptable inert carrier such as ethanol, glycerol, water, and the like, Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. En general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in this field.

The agents of the instant invention may also be administered in intranasal form via use of suitable intranasal vehicles, or via transdermal routes, using those forms of transdermal skin patches well known to those of ordinary skill in that art. To be administered in the form of a transdermal delivery system, the dosage administration will generally be continuous rather than intermittent throughout the dosage regimen.

Parenteral and intravenous forms may also include minerals and other materials to make them compatible with the type of injection or delivery system chosen.

Antibodies and fragments thereof, as agents of the inventions described herein, may be administered by any of the methods of administering antibodies known in the art including by intravenous, intramuscular and subcutaneous methods, including by injection or infusion, and can be introduced directly into the site of the cancer.

Gβγ is a beta-gamma complex composed of the heterotrimeric G proteins Gβ (beta subunit) and Gγ (gamma subunit) that are closely bound to one another.

The subject can be human. In an embodiment of the invention described herein, the Gβγ is a mammalian Gβγ. In a preferred embodiment, the Gβγ is a human Gβγ. In an embodiment, the p110β is a mammlian p110β. In a preferred embodiment, the p110β is a human p110β. in art embodiment the p110β has the sequence of RefSeq Accession No. NM_{—}006219.1. in an embodiment the p110β has the sequence:

| | | |
|---|---|---|
| 1 | MCFSFIMPPA MADILDIWAV DSQIASDGSI PVDFLLPTGI YIQLEVPREA TISYIKQMLW | (SEQ ID NO: 1) |
| 61 | KQVHNYPMFN LLMDIDSYMF ACVNQTAVYE ELEDETRRLC DVRPFLPVLK LVTRSCDPGE | |
| 121 | KLDSKIGVLI GKGLHEFDSL KDPEVNEFRR KMRKFSEEKI LSLVGLSWMD WLKQTYPPEH | |
| 181 | EPSIPENLED KLYGGKLIVA VHFENCQDVF SFQVSPNMNP IKVNELAIQK RLTIHGKEDE | |
| 241 | VSPYDYVLQV SGRVEYVFGD HPLIQFQYIR NCVMNRALPH FILVECCKIK KMYEQEMIAI | |
| 301 | EAAINRNSSN LPLPLPPKKT RIISHVWENN NPFQIVLVKG NKLNTEETVK VHVRAGLFHG | |
| 361 | TELLCKTIVS SEVSGKNDHI WNEPLEFDIN ICDLPRMARL CFAVYAVLDK VKTKKSTKII | |
| 421 | NPSKYQTIRK AGKVHYPVAW VNTMVFDFKG QLRTGDHLII SWSSFPDELE EMLNPMGTVQ | |
| 481 | TNPYTENATA LHVKFPENKK QPYYYPPFDK IIEKAAEIAS SDSANVSSRG GKKFLPVLKE | |
| 541 | ILDRDPLSQL CENEMDLIWT LRQDCREIFP QSLPKLLLSI KWNKLEDVAQ LQALLQIWPK | |
| 601 | LPPREALELL DFNYPDQYVR EYAVGCLRQM SDEELSQYLL QLVQVLKYEP FLDCALSRFL | |
| 661 | LERALGNRRI GQFLFWHLRS EVHIPAVSVQ FGVILEAYCR GSVGHMKVLS KQVEALNKLK | |
| 721 | TLNSLIKLNA VKLNRAKGKE AMHTCLKQSA YREALSDLQS PLNPCVILSE LYVEKCKYMD | |
| 781 | SKMKPLWLVY NNKVFGEDSV GVIFKNGDDL RQDMLTLQML RLMDLLWKEA GLDLRMLPYG | |
| 841 | CLATGDRSGL IEVVSTSETI ADIQLNSSNV AAAAAFNKDA LLNWLKEYNS GDDLDRAIEE | |
| 901 | FTLSCAGYCV ASYVLGIGDR HSDNIMVKKT GQLFHIDEGH ILGNFKSKFG IKRERVPFIL | |
| 961 | TYDFIHVIQQ GKTGNTEKFG RFRQCCEDAY LILRRHGNLF ITLFALMLTA GLPELTSVKD | |
| 1021 | IQYLKDSLAL GKSEEEALKQ FKQKFDEALR ESWTTKVNWM AHTVRKDYRS | |

(Underlined region shows 24 amino acid residues required for p110β activation by Gβγ).

Compositions are provided comprising a peptide comprising amino acid residues having the KAAEIASSDSANVSSRGGKKFLPV (SEQ ID NO:6). In an embodiment the composition is a pharmaceutical composition. In an embodiment, the composition or pharmaceutical composition comprises a pharmaceutically acceptable carrier.

All combinations of the various elements described herein are within the scope of the invention unless otherwise indicated herein or otherwise clearly contradicted by context.

This invention will be better understood from the Experimental Details, which follow. However, one skilled in the art will readily appreciate that the specific methods and results discussed are merely illustrative of the invention as described more fully in the claims that follow thereafter.

### Experimental Details

### Introduction

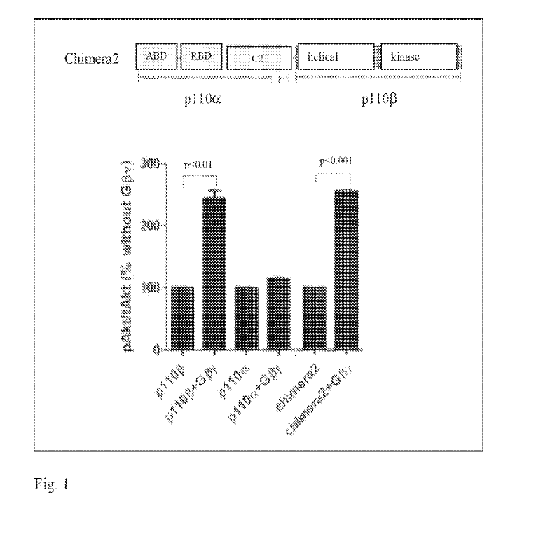

A great deal of progress has been made in defining the mechanism of p110α/δ regulation by RTKs. In contrast, regulation of p110β and p110γ by GPCRs is not well understood. Both subunits are activated by directly binding to Gβγ. For p110γ, the binding site involves both the N- and C-termini of p110γ, suggesting an extensive interaction surface. In contrast, it is shown herein that Gβγ fully activates a chimeric protein containing the N-terminal half of p110α (the ABD, RBD and C2 domains) and only the helical and kinase domains form p110β (FIG. 1). Based on these data, analysis of Gβγ interactions restricted to the helical and kinase domains of p110β was pursued.

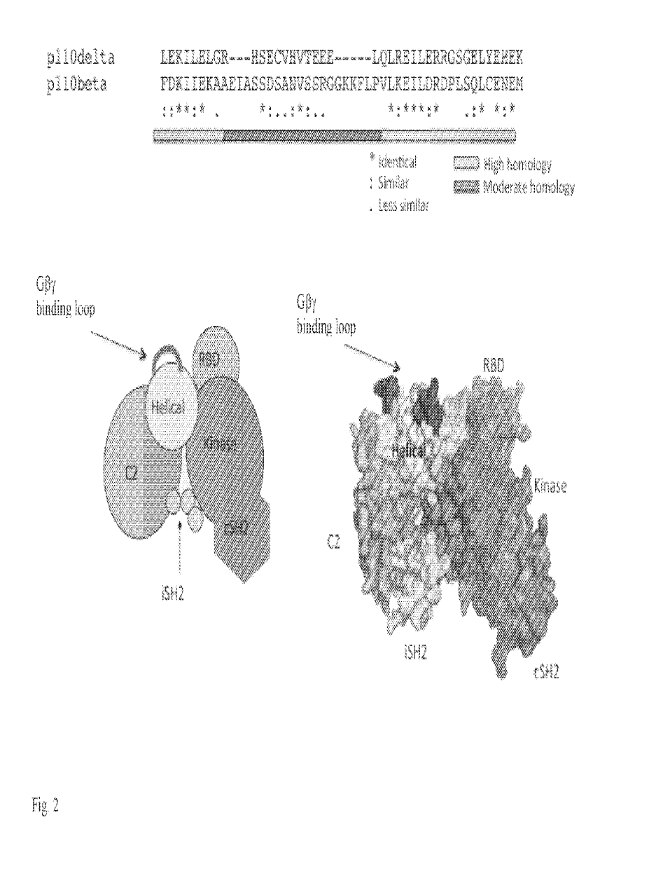

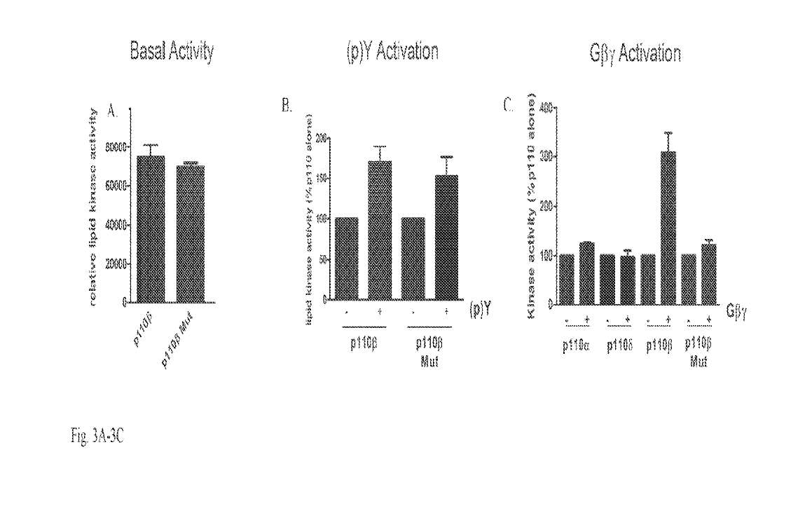

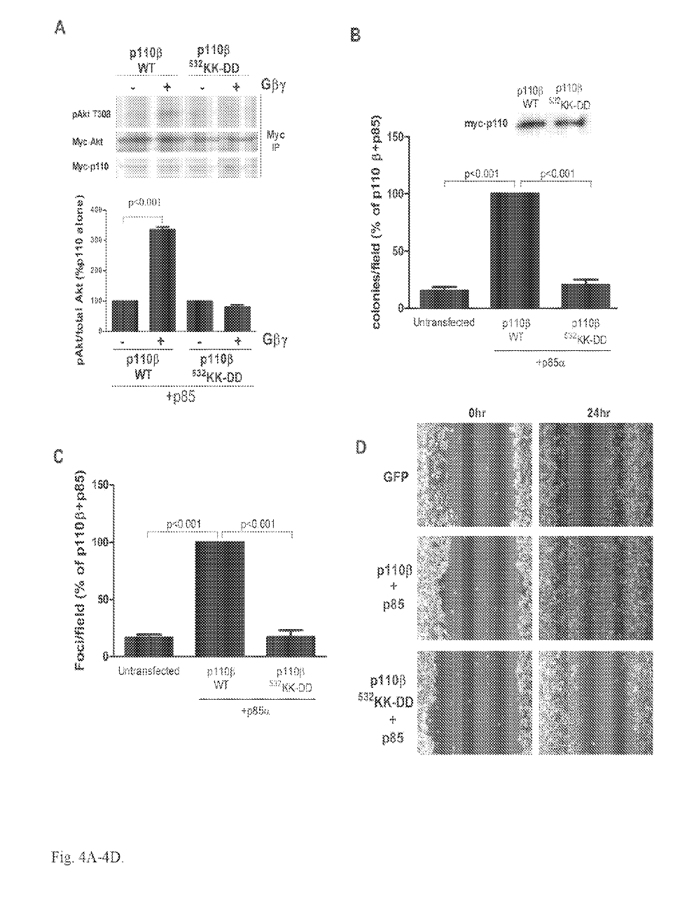

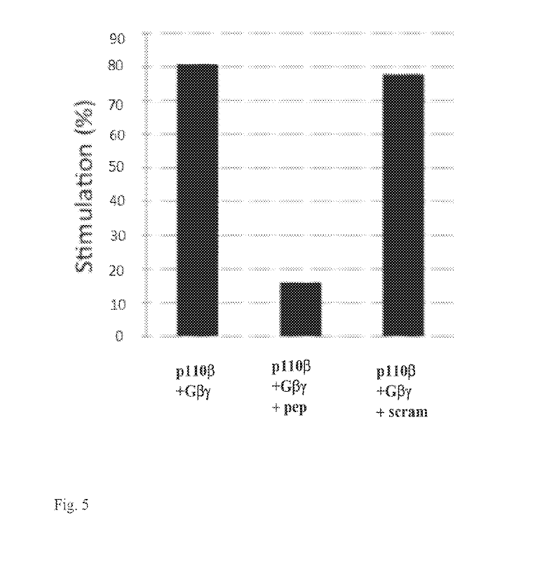

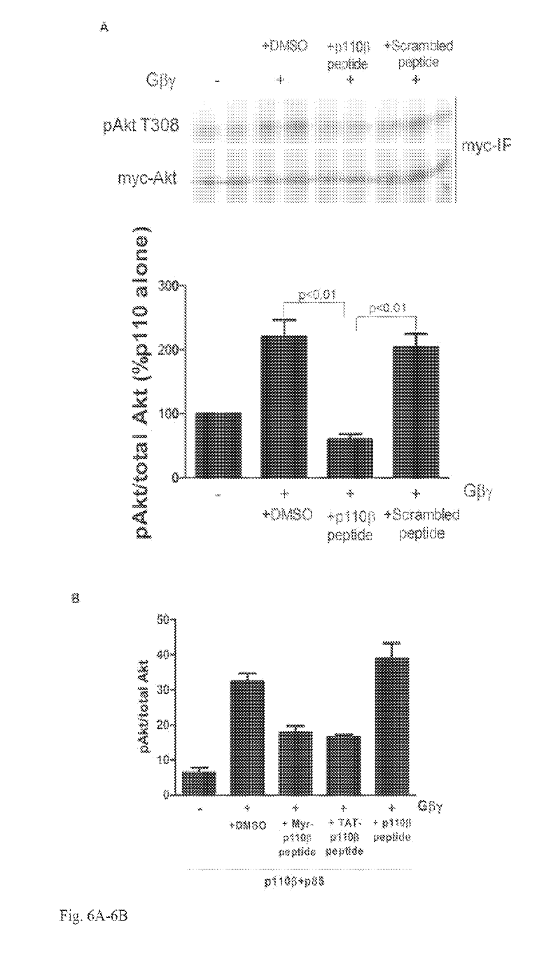

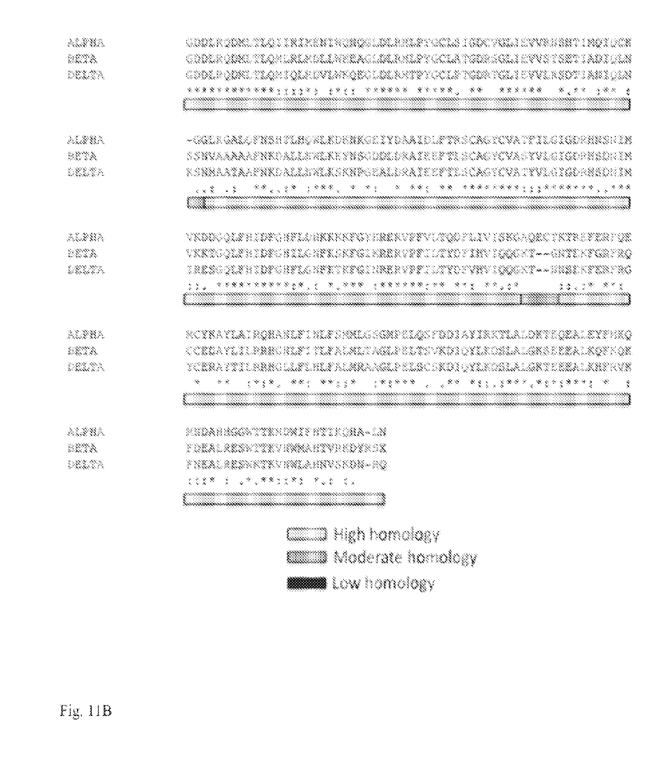

A comparison of the helical and kinase domains of p110α, β and δ shows a high degree of similarity (FIG. 11), A notable region of non-conservation occurs in the C2 domain-helical domain linker (FIG. 2, top). This loop is predicted to be surface accessible, but is only partially observed in the p110β crystal structure, presumably due to its flexibility. To test whether this loop might be involved in Gβγ binding, the corresponding loop from p110δ was substituted into intact p110β. The resulting mutant shows normal basal PI 3-kinase activity, and is normally regulated by tyrosine phosphopeptides, but shows no activation in the presence of Gβγ (FIG. 3). Similar results were obtained with by mutating two highly conserved residues in the loop, ⁵³²KK-DD (data not shown). Importantly, the ⁵³²KK-DD construct also showed decreased Gβγ-stimulated p110β signaling in vivo; Gβγ activation of Akt, formation of colonies in soft agar, formation of foci, and cell migration in a wound healing assay, were robust in cells expressing p85 plus wild type p110β, but deficient in cells expressing p85 plus mutant p110β (FIG. 4A-D). These data suggest that the transforming activity of p110β depends on its regulation by Gβγ.

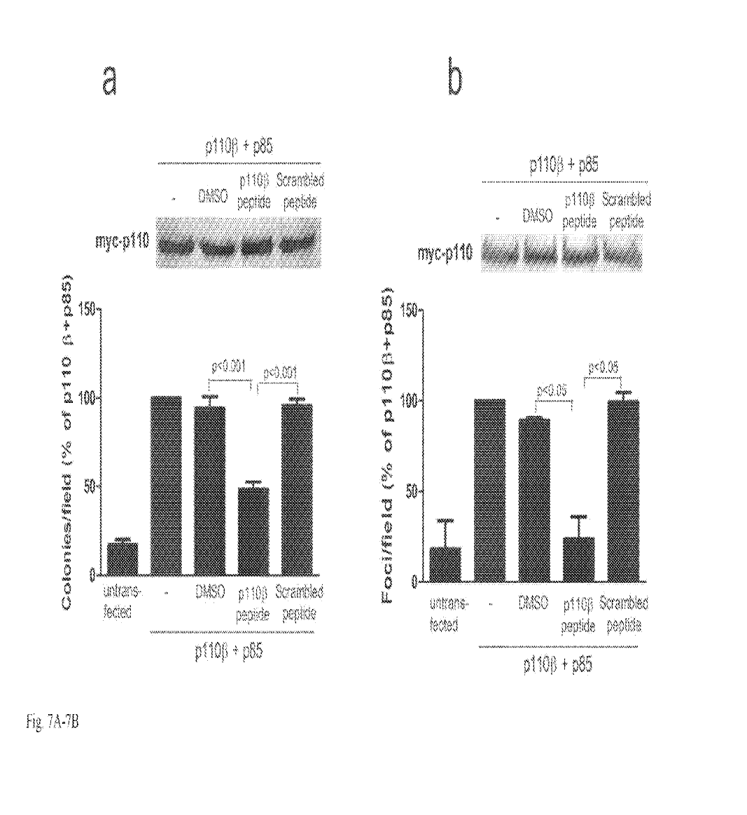

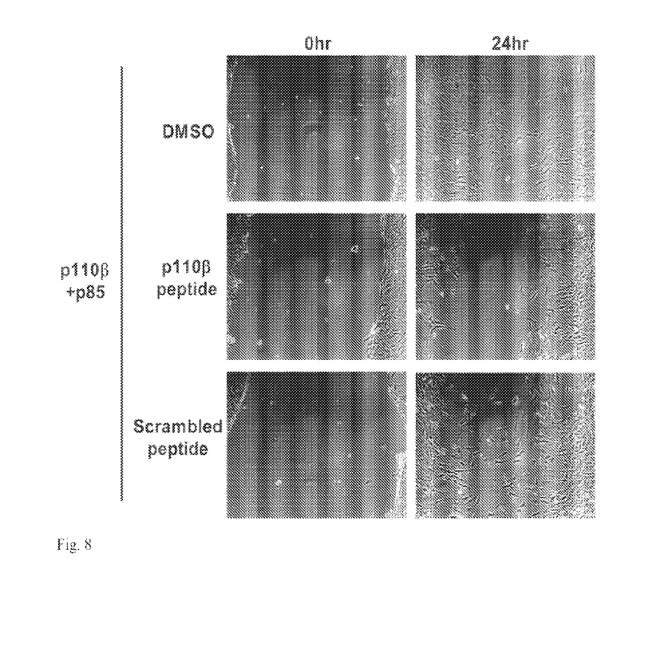

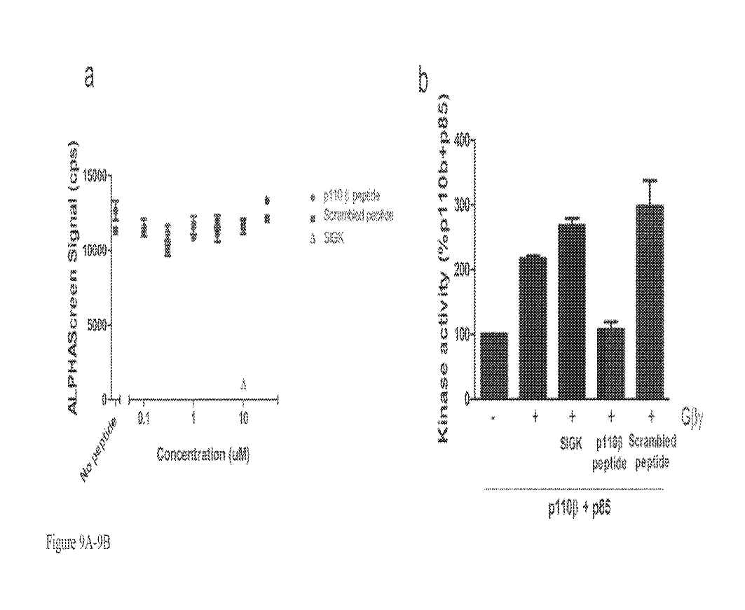

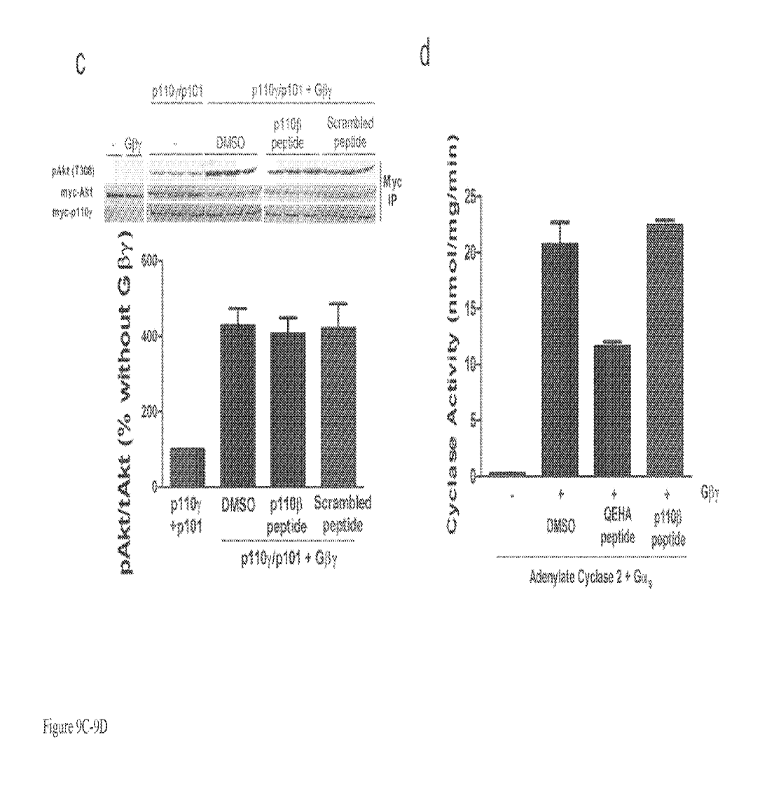

To test the possibility that small molecules targeting the p110β-Gβγ interface could be used as therapeutics, a peptide was designed derived from the Gβγ-binding loop in p110β. having the sequence KAAEIASSDSANVSSRGGKKFLPV (SEQ ID NO:6) a scrambled version of the peptide serves as a negative control. Incubation of the peptide with p110β (5-fold in excess of Gβγ) markedly reduces activation of p110β by Gβγ, whereas the scrambled peptide has no effect (FIG. 5). The test whether the peptide could be efficacious in vivo, we created a cell permeable myristylated version of the peptide. The peptide blocked activation of Akt in cells transfected with Gβγ (FIG. 6A); the peptide's inhibitory activity required its entry into the cells, since both myristoylated and TAT-tagged peptide inhibited Gβγ activation of Akt, whereas unlabeled peptide had no effect (FIG. 6B). The myristoylated peptide blocked the formation of colonies in soft agar (FIG. 7A) and the formation of foci (FIG. 7B), both measures of transformation, in NIH3T3 cells transfected with p85/p110β. The peptide also blocked the enhanced migration of NIH3T3 cells transfected with p110β/p85 in a wound healing assay (FIG. 8).

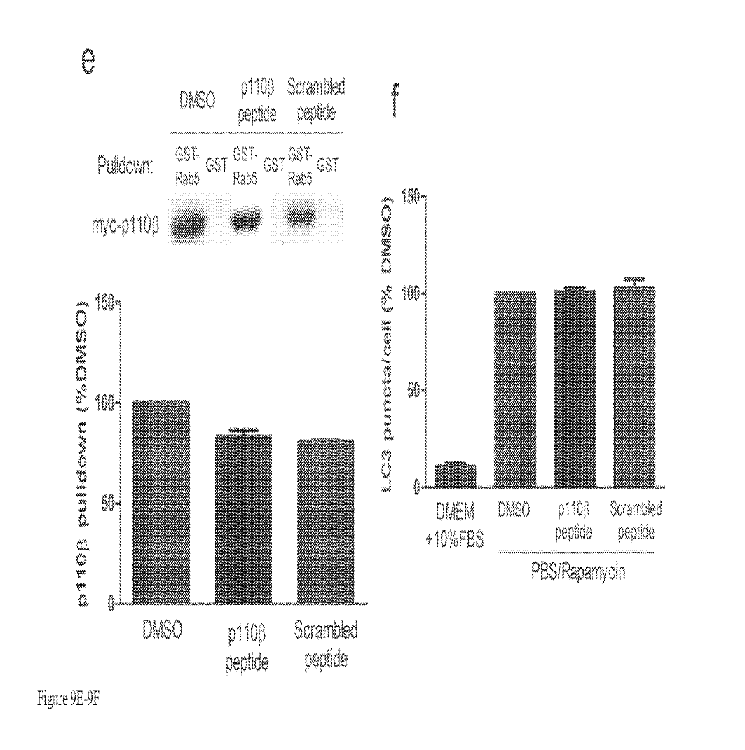

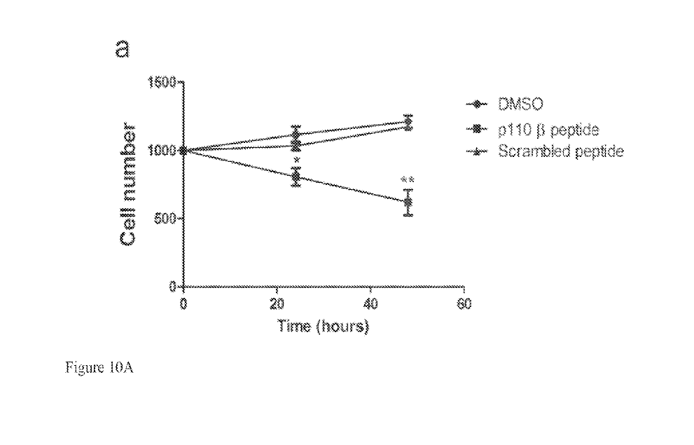

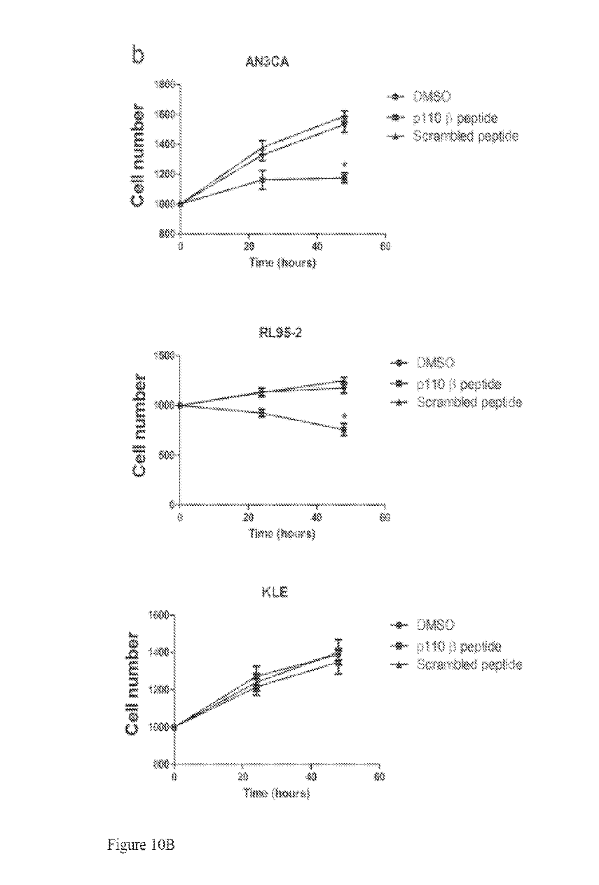

Control experiments showed that the effects of the peptide are specific for p110β-Gβγ interactions. The peptide did not reduce p110β expression (FIG. 7), and it did not compete with the binding to Gβγ of a previously characterized SIGK peptide that targets the Gβγ hotspot (FIG. 9A) [25]. In the reciprocal experiment, the SIGK peptide did not inhibit activation of p85/p110β by Gβγ in vitro (FIG. 9B., These data suggests that the SIGK peptide and the p110β peptide bind to distinct sites on Gβγ. Consistent with this finding, the p110β peptide had no effect on Gβγ-dependent activation of the Class IB PI3K p101/p110γ (FIG. 9C) or the synergistic activation of adenylyl cyclase by Gβγ and Gαs (FIG. 9D). Similarly, the peptide had no effect on p110β binding to Rab5 or on the p110β-dependent induction of autophagy [26] (FIG. 9E, 9F). Thus, the effects of the myristoylated peptide are specific for the disruption of p110β-Gβγ interactions.

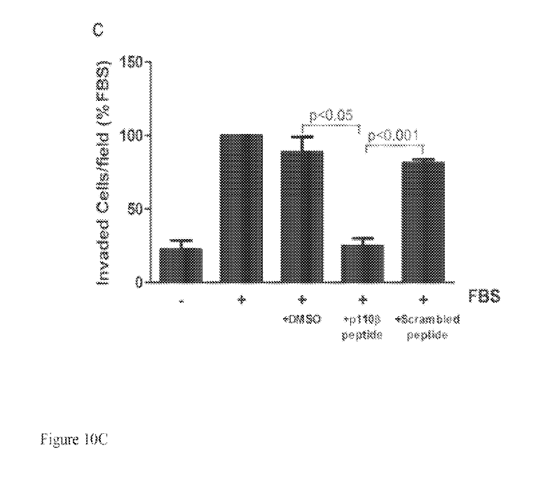

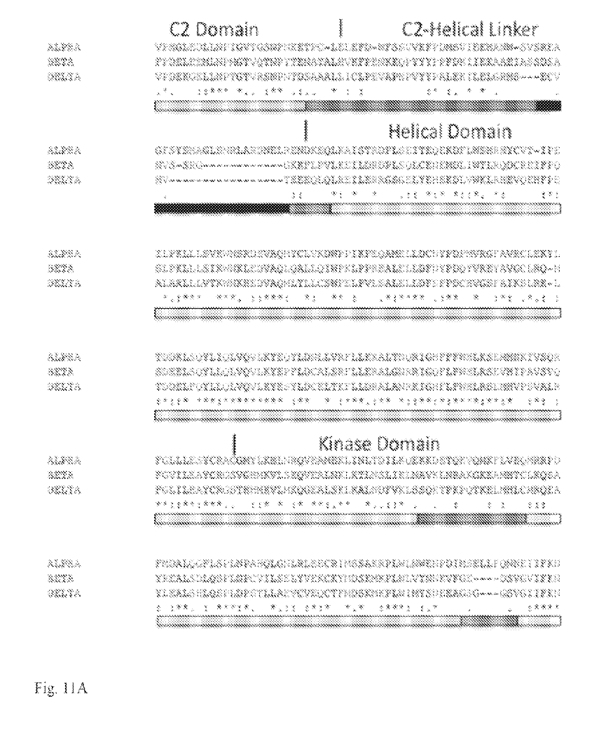

Finally, the peptide was evaluated for effects on the growth of PC3 prostate cancer cells, which are known to require p110β for growth. Incubation of PC3 cells with the peptide, but not a scrambled control, caused a decrease in PC3 cells number, suggesting that the peptide was cytotoxic rather than cytostatic (FIG. 10A). Similar effects were seen in the PTEN-null endometrial cancer cell lines AN3CA and RL95-2, but not in the PTEN positive endometrial cancer line KLE (FIG. 10B). Myristoylated p110β-derived peptide also inhibited PC3 cells chemotaxis toward serum in a Boyden chamber assay (FIG. 10C). Importantly, published studies have shown that kinase-dead p110β can rescue cell growth in cell lines where proliferation is inhibited by p110β knockout. Since currently available p110β inhibitors target the active site of p110β and act by inhibiting its kinase activity, they would not be expected to suppress the growth of cells that depend on p110β for growth. In contrast, the p110β peptide described here inhibits p110β by a distinct mechanism, and is likely to be more efficacious at suppressing the growth of p110β-dependent prostate cancer cells than active site inhibitors.

For a gene therapy approach the cDNA for a stable inert small protein such as thioredoxin or SUMO is modified so as to include the p OP peptide sequence at its N- and C-termini. If needed, additional copies of the p110β peptide sequence can be inserted as extensions of surface loops, based on the crystal structures of these proteins. In all cases, stability of the peptide-protein fusion in vitro (by NMR) and in vivo (by protein half-life), and inhibition of Gβγ-p110β interactions in vitro and in vivo will be tested. Such a reagent can be introduced into cells by transfection or via recombinant adenoviral or lentiviral vectors, and expands the option for the delivery of a reagent that disrupts p110β-Gγ interactions in vivo.

Accordingly, a 24-amino acid surface-exposed region of p110β has been identified that is required for its activation by Gβγ. Mutation of this binding site abolishes Gβγ activation of p110β in vitro and in vivo, and greatly decreases the ability of p110β to induce the transformation of NIH 3T3 cells. A peptide derived from the Gβγ-binding site in p110β blocks Gβγ activation of p110β and a cell-permeant version blocks Akt activation and foci formation, and causes cell death in PC3 prostate cancer cells. Recombinant carrier proteins containing multiple copies of the p110β peptide sequence can be used in a gene therapy approach. Peptides derived from the Gβγ-binding site in p110β can be peptidomimetic inhibitors of p110β-Gβγ interactions, and efficacious in treating cancer, inflammatory disease and other human disorders.

### REFERENCES

1. Davis, T. L., Bonacci, T. M., Sprang, S. R., and Smrcka, A. V. (2005) Structural and molecular characterization of a preferred protein interaction surface on G protein beta gamma subunits. Biochemistry 44, 10593-10604
2. Dou. Z., Chattopadhyay, M., Pan, J. A., Guerriero, J. L., Jiang, Y. P., Ballou, L. M., Yue, Z., Lin, R. Z., and .Zone, W. X. (2010) The class IA phosphatidylinositol 3-kinase p110-beta subunit is a positive regulator of autophagy. J. Cell Biol. 191, 827-843
3. Wee, S., Wiederschain, D., Maira, S. M. Loo, A., Miller, C. deBeaumont, R., Stegmeier, F., Yao, Y. M,, and Lengauer, C. (2008) PTEN-deficient cancers depend on PIK3CB. Proc. Natl. Acad. Sci. U. S. A. 105, 13057-1:3062
4. Bookout, A. L., Finney A. E., Guo R., Peppel, K., Koch, W. J., and Daaka. Y. (2003) Targeting Gbetagamma signaling to inhibit prostate tumor formation and growth. J Biol. Chem. 278, 37569-37573
5. Berenjeno, I. M. Guillermet-Guibert, J., Pearce, W. Gray, A., Fleming, S., and Vanhaesebroeck, B. (2012) Both p110alpha and p110beta isoforms of P13K can modulate the impact of loss-of-function of the PTEN tumour suppressor. Biochem. J. 442, 151-159
6. Chen, J., DeVivo, M., Dingus, J., Harry, A., Li, J., Sui, J., Carty, D. J., Blank, J. L., Exton, J. H., Stoffel, R. H., and et al. (1995) A region of adenylyl cyclase 2 critical for regulation by G protein beta gamma subunits Science 268. 1166-1169

1. A method of treating a disease in a subject comprising administering to the subject an amount of an agent which reduces, or prevents, interaction of a Gβγ with a p110β effective to treat the disease.

2. The method of claim 1, wherein disease is a cancer.

3. The method of claim 1, wherein the agent is a peptide comprising amino acid residues having the same sequence as residues 513 to 537 of SEQ ID NO:1, or is an active portion of residues 513 to 537 of SEQ ID NO:1.

4. The method of claim 3, wherein the peptide or active portion is acylated or is myristoylated.

5. The method of claim 1, wherein the agent is an oligonucleotide which reduces binding of the Gβγ to the p110β or blocks the binding of the Gβγ to the p110β.

6. The method of claim 1, wherein the agent is an aptamer, a nucleic acid, an oligonucleotide, a small organic molecule of 2000 Daltons or less, or a nucleic-acid effector of RNAi.

7. The method of claim 3, wherein the peptide is 30 amino acids or less in length.

8. The method of claim 1, wherein the agent comprises a cDNA encoding a first portion comprising a stable inert protein, and encoding a second portion comprising
(i) (a) a peptide having the sequence of residues 513 to 537 of SEQ ID NO:1 or (b) a peptide having the sequence KAAEIASSDSANVSSRGGKKFLPV (SEQ ID NO:6), wherein the second portion is attached via a peptide bond to the C-terminus of the stable inert protein, or to the N-terminus of the stable inert protein,
or
(ii) (a) a peptide having the sequence of residues 513 to 537 of SEQ ID NO:1 or (b) a peptide having the sequence KAAEIASSDSANVSSRGGKKFLPV (SEQ ID NO:6), wherein the second portion is attached to each of the C-terminus and N-terminus of the stable inert protein.

9. The method of claim 8, wherein the agent is introduced into cells of the subject by a technique comprising transduction, lentiviral delivery or adenoviral delivery.

10. The method of claim 8, wherein the stable inert protein is thiredoxin or small ubiquitin-like modifier (SUMO).

11. (canceled)

12. The method of claim 2, wherein the cancer is a c-Kit-dependent cancer and is a testicular cancer.

13. The method of claim 2, wherein the cancer is a prostate cancer or a glioblastoma, and is phosphatase and tensin homolog (PTEN) null.

14. The method of claim 1, wherein the agent binds to the C2 domain helical linker of p110β.

15. The method of claim 1, wherein the agent binds to a portion of Gβγ which binds to the C2 domain helical linker of p110β.

16. The method of claim 15, wherein the agent is a peptide comprising amino acid residues having the same sequence as residues 513 to 537 of SEQ ID NO:1 or has the sequence KAAEIASSDSANVSSRGGKKFLPV (SEQ ID NO:6).

17-18. (canceled)

19. A method for identifying an agent as an inhibitor of Gβγ activation of p110β comprising contacting a p110β with the agent in the presence of Gβγ under conditions permitting the Gβγ to activate the p110β and quantifying activation of p110β by the Gβγ, wherein reduced activation of p110β by Gβγ in the presence of the agent as compared to activation of p110β by Gβγ in the absence of the agent indicates that the agent is an inhibitor of Gβγ activation of p110β.

20-21. (canceled)

22. A method for inhibiting Gβγ activation of p110β without inhibiting lipid kinase activity of p110β comprising contacting the p110β with an agent that reduces or prevents interaction of Gβγ with the p110β without inhibiting lipid kinase activity of p110β.

23. The method of claim 22, wherein the p110β contacted with the Gβγ is activatable by receptor tyrosine kinases.

24. The method of claim 22, wherein the agent is a peptide comprising amino acid residues having the KAAEIASSDSANVSSRGGKKFLPV (SEQ ID NO:6).

25-30. (canceled)

31. A method of identifying an inhibitor of interaction between a Gβγ and a p110β, the method comprising a) modeling in silico the 3-dimensional site or sites on Gβγ which bind KAAEIASSDSANVSSRGGKKFLPV (SEQ ID NO:6), b) testing in silico if a compound from a library of compounds binds to the modeled 3-dimensional site or sites, and c) determining in vitro if a chemically stable small molecule identified as binding to the site or sites in silico in b) inhibits the interaction between a Gβγ and a p110β, wherein a chemically stable small molecule that inhibits the interaction between a Gβγ and a p110β is identified as an inhibitor.

32-34. (canceled)
